(19) United States
(12) Patent Application Publication
(10) Pub. No.: US 2026/0105416 A1
(43) Pub. Date: Apr. 16, 2026

(54) SINGLE IDENTIFIER BENEFITS DISTRIBUTION

(71) Applicant: PRESCRYPTIVE HEALTH, INC., Redmond, WA (US)

(72) Inventors: Christopher Scott Blackley, Woodinville, WA (US); Kevin C. Young, Sisters, OR (US); Marc Gillette, Redmond, WA (US); Allen Edward Nieman, Redmond, WA (US)

(21) Appl. No.: 18/912,513

(22) Filed: Oct. 10, 2024

(51) Int. Cl. G06Q 10/1057; G16H 40/20

(52) U.S. Cl. CPC G06Q 10/1057; G16H 40/20

(57) ABSTRACT

A client identifier may be mapped to identifiers corresponding to a set of providers according to conditions defined by arrangements between the client and the set of providers. The mapping may be used to compare one or more values provided by the set of providers, where the one or more values may correspond to a requestable perquisite requested by the client, and a provider may be selected from the set of providers based, at least in part, on the comparison.

### BACKGROUND

Employer-based benefits plans, such as health insurance and prescription benefits, typically involve various identification numbers, including policy, subscriber, member identification, and prescription claim routing numbers. These plans often have intricate rules governing coverage, making certain plans more advantageous for specific benefits. When employers offer customized benefits from multiple insurers or provide their own coverage, employees receive multiple benefits card numbers, leading to confusion and difficulty in identifying the most advantageous number to use for a given transaction. This confusion can result in higher costs for both the individual and employer. Additionally, when employers switch plans, the new identification numbers are not always promptly communicated to benefits providers, causing delays and frustration for both the providers and the individuals.

### BRIEF DESCRIPTION OF THE DRAWINGS

Various techniques will be described with reference to the drawings, in which:

FIG. 1 illustrates an example of an architecture for a process to access benefits provided by benefit processors, in accordance with an embodiment;

FIG. 2 illustrates an example of a mapping generated at a customer module of the architecture of FIG. 1, in accordance with an embodiment;

FIG. 3 illustrates an example of a relationship mapping used at a claims module of the architecture of FIG. 1, in accordance with an embodiment;

FIG. 4 illustrates an example of a process for accessing benefits provided by benefit processors, in accordance with an embodiment; and

FIG. 5 illustrates a computing device that may be used in accordance with at least one embodiment or an environment in which various embodiments can be implemented.

### DETAILED DESCRIPTION

Techniques and systems described below relate to a platform for a client to be assigned a single identifier that allows the client, when requesting access to a requestable perquisite, such as a benefit, to automatically obtain an optimized response in a streamlined manner. In particular, the techniques and systems described below provide a permanently assigned prescription benefits card account number that remains active regardless of the pharmacy benefits manager (PBM), which simplifies the process for employers and employees, reduces confusion, and allows for the seamless integration of various prescription benefits, ultimately leading to cost savings and improved adherence to prescribed medications. In at least one embodiment, the platform includes generating a client identifier for a client and, in response to receiving a request for the client to access a requestable perquisite, generating a mapping of the client identifier to one or more identifiers corresponding to a set of providers able to meet the request. The platform further includes inputting, into the mapping, values from the set of providers associated with the requestable perquisite, selecting a provider from the set of providers based, at least in part, on the mapping and preset instructions, and providing the request to the selected provider to obtain a response from the selected provider. The response is provided from the selected provider as a result of processing the received request.

This minimizes a burden on the client to select from among multiple benefit providers using a single identifier that is assigned to the client. In one example, the client may be a payor or employer obtaining health-related benefits to be provided to associated payees or employees, where the payees or employees may also be clients. Agreements, e.g., documents such as contracts, may be established between the payor and various health benefit providers (e.g., organizations processing health benefit claims, also referred to as benefit processors), which may include health insurance providers and/or pharmacy benefit managers (PBMs). The agreements may be arrangements between the payor and the benefit processors that define one or more sets of conditions for the arrangements. Conventionally, a separate identifier may be used for each contract between the payor and a benefit processor to identify specific benefits provided to the payor through the contract. An identifier may be, as one example, a benefit identification number/processor control number (BIN/PCN) pair that corresponds to a contract between the payor and a benefit processor. The BIN may identify a benefit provider and the PCN may indicate a specific group within the benefit processor. For example, the PCN may be used to differentiate between different plans or programs offered by a benefit processor.

The documents defining terms and conditions of arrangements (e.g., contracts) may correspond to different aspects of health insurance and/or healthcare provisions and define specific pricing and services offered through the particular contract. In order for a payor or payee to access a desired perquisite, or benefit, the payor or payee may be required to select an identifier from multiple identifiers. It may be challenging, however, for the payor or payee to determine which BIN/PCN pair may provide the most suitable option for obtaining the desired benefit, such as the highest availability and/or lowest price for the desired benefit. As a result, the payor or payee may select a BIN/PCN pair that does not represent an optimal selection from an available set of BIN/PCN pairs.

This may be mitigated, as described herein, by a computing platform, such as a profile mapping system, that assigns a single identifier to a client (e.g., a client identifier) and maps the identifier to multiple contracts established between the payor and benefit processors. In at least one embodiment, the client identifier may be connected to multiple other identifiers and may hereafter be referred to as a nexus identifier. The benefit processors may include intermediaries that process requests to access benefits available to a client, or payor, and adjudicate, moderate, and distribute (e.g., provide access to) the benefits. In at least one embodiment, the benefit processors (e.g., benefit providers) may include PBMs and health insurance providers, as discussed above. By mapping the nexus identifier to multiple contracts, the nexus identifier may be linked to various benefit processors, and to groups or subdivisions within a benefit processor in an automated manner. When a claim is submitted requesting access to a benefit, all contracts relevant to that benefit may be identified and compared to identify a benefit processor, and group or subdivision therein, that provides the benefit with criteria optimized for the requester (e.g., the payor or payee requesting the benefit). The payor and/or payee may thereby identify a benefit provided through a benefit processor that satisfies criteria that may be specified by the payor, such as pricing, copay assistance, and other financial options.

Moreover, the nexus identifier may be constant, unchanged, and usable (e.g., active), during subsequent modifications to a client's benefits profile. For example, the client's benefit profile may include a network of benefit processors with which the client may have established arrangements. As an example, an arrangement may include one or more contracts that define terms and conditions of the arrangement. The arrangement may be used as a reference to determine how a benefit may be distributed or provided to the client via a provider of the benefit (e.g., a pharmacy) that is to dispense the benefit to the client. After initial onboarding of the client to the computing platform, the client's benefits profile may change. For example, new arrangements may be added and/or arrangements may be terminated. The nexus identifier, however, remains assigned to the client, despite changes to a mapping of the nexus identifier to benefit processor identifiers. The nexus identifier, therefore, is robust to updates to the client's benefit profile and thereby reduces an amount of effort of the client to efficiently access benefits available to the client.

In the preceding and following description, various techniques are described. For purposes of explanation, specific configurations and details are set forth in order to provide a thorough understanding of possible ways of implementing the techniques. However, it will also be apparent that the techniques described below may be practiced in different configurations without the specific details. Furthermore, well-known features may be omitted or simplified to avoid obscuring the techniques being described.

Techniques described and suggested in the present disclosure improve the field of computing, especially benefit routing, identification, and adjudication, by assigning a nexus identifier to a payor that captures terms and conditions of agreements established between a payor and benefit processor. Additionally, techniques described and suggested in the present disclosure improve the efficiency of computing systems by performing identification of all agreements relevant to a payor request and comparison of attributes of the agreements in real time. Moreover, techniques described and suggested in the present disclosure are necessarily rooted in computer technology in order to overcome problems specifically arising with managing multiple benefits offered by multiple benefit processors by implementing algorithms to automatically map available benefits and benefit processors to a given client and streamline identification of target variables among the available options.

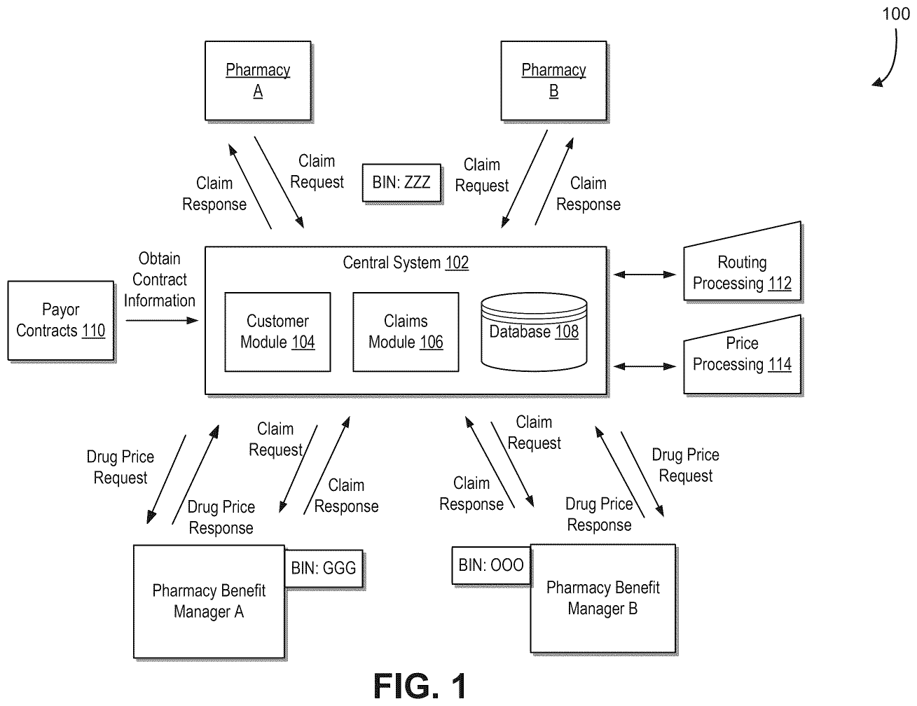

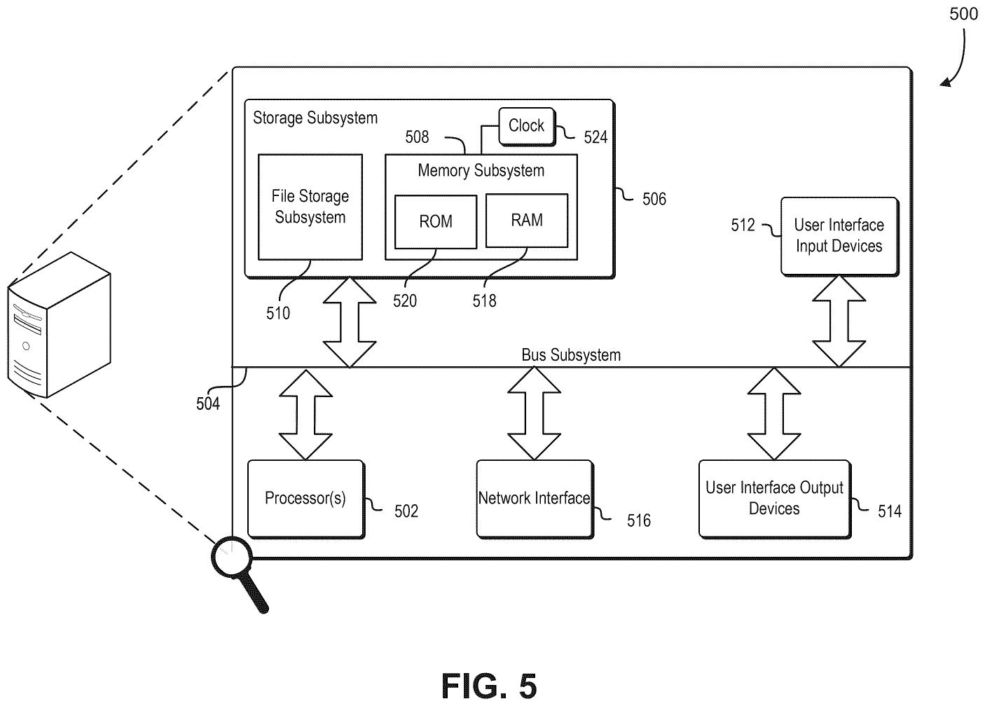

FIG. 1 illustrates an example architecture 100 for a profile mapping system. In at least one embodiment, the profile mapping system may be utilized to access requestable perquisites or items that may be adjudicated, moderated, and/or distributed by benefit processors. In at least one embodiment, the requestable perquisites may be benefits associated with healthcare. As shown in FIG. 1, in at least one embodiment, the architecture 100 may include exchange of information between a central system 102 of the profile mapping system and a plurality of entities. As an example, the central system 102 may be one or more computing devices, such as a computing device 500 illustrated in FIG. 5, that may be controlled and operated by a management entity or third party that is separate from a payor (e.g., a client) and the plurality of entities linked to the payor. In another example, the central system 102 may be one or more computing devices controlled and operated by a benefit processor, such as a health insurance provider or a PBM.

In at least one embodiment, the central system 102 of the profile mapping system may be cloud-hosted to provide a high trust and high security environment for receiving, storing, and processing confidential information. In yet another embodiment, the central system 102 may utilize one or more models, such as machine learning models and relational models, to perform at least a portion of the operations included in the retrieval, parsing, and/or analysis of information implemented at the central system 102.

In at least one embodiment, the central system 102 may include a first component (e.g., a customer module 104), a second component (e.g., claims module 106), and a third component (e.g., database 108). The customer module 104 and the claims module 106 may each include, for example, one or more software programs (e.g., algorithms) including commands and instructions for receiving data corresponding to a request and performing operations to process the request and corresponding data and to generate a result based on the request and processed data. In at least one embodiment, the customer module 104 and the claims module 106 may be implemented at one or more hardware components, e.g., computing resources, of the central system 102 that may be dedicated to performing tasks and operations associated with each module. The customer module 104 may include hardware configured to carry out operations to, for example, receive requests input to the central system 102, retrieve relevant data from the database 108, and access benefit processors to obtain offers from the benefit processors based on the requests. At the customer module 104, the offers may further be compared according to rules and criteria specified by the claims module 106 algorithms, and a benefit processor and associated offer may be selected based on the comparison.

In at least one embodiment, the claims module 106 may include hardware configured to carry out operations to receive the selected benefit processor and offer and process the request using the selected benefit processor and offer. In at least one embodiment, processing the request may include exchanging information with the selected benefit processor and confirming the offer. The database 108 may store data that may be commonly used among all payor/benefit processor correlations or relationships. As one example, the database 108 may store pricing information that may be fixed for one or more correlations (e.g., relationships defined by agreements or contracts) between a payor and benefit processors. By incorporating the customer module 104, the claims module 106, and the database 108 in the central system 102, the central system 102 may efficiently retrieve and parse large quantities of data from different agreements between a payor and various benefit processors and return outcomes based on requests input thereto. In at least one embodiment, the central system 102 may allow a nexus identifier identifying a payor to be linked to multiple identifiers corresponding to benefit processors and offers provided by the benefit processors.

In at least one embodiment, to generate a response to a request to identify a benefit processor with an offer that satisfies one or more target criteria, terms and conditions of relationships between a payor and various benefit processors may be input to the customer module 104. For example, as depicted in FIG. 1, payor contracts 110 may be input to the central system 102. In at least one embodiment, the payor contracts 110 may include electronic documents in text or other formats that may be entered into the central system 102 by a user. The payor contracts 110 may be input directly to the central system 102 through, for example, a user interface of the central system 102, or may be transmitted to the central system 102 from another computing device. In at least one embodiment, the payor contracts 110 may be input to the central system 102 and received by the customer module 104 independent of requests (e.g., claim requests) provided to the central system 102. For example, the payor contracts 110 may be input to the central system 102 by the user whether or not a claim request is received at the central system 102, and the payor contracts 110, or data extracted from the payor contracts 110, may be stored in the database 108.

In at least one embodiment, the central system 102 may receive the payor contracts 110 during an onboarding process to allow the payor to be recognized and identified by the central system 102. Furthermore, the onboarding process may allow relationships, e.g., contractual agreements, between the payor and benefit processors, such as entities providing benefit plans, to be recorded at the central system 102 (e.g., stored at the database 108) as a profile of the payor, which may be used for subsequent reference and retrieval. During the onboarding process, the payor may be assigned a nexus identifier, such as a BIN/PCN pair, that is unique and used exclusively to identify the payor. Moreover, the nexus identifier may be permanently assigned and durable such that the payor does not need to obtain a new identifier over time. For example, as indicated in FIG. 1, a BIN portion of the identifier assigned to the payor may be ZZZ.

Once the payor is onboarded to the central system 102, the relationships between the payor and benefit processors may be subsequently updated. For example, if a new contract is established between payor and a benefit processor, the payor may notify the central system 102 by providing the new contract to the central system 102, which may then be added to the payor's profile. Similarly, if an existing payor contract is terminated, the payor may notify the central system 102 to request that the contract be removed from the payor's profile. Regardless of how the payor's profile is updated, the nexus identifier of the client remains active.

In at least one embodiment, as shown in FIG. 1, one or more requests, e.g., claim requests, may be received from one or more of the plurality of entities, which may be representatives of the payor to the benefit processors and may be providers of a benefit (e.g., requestable perquisite) to the payor. For example, the providers or representatives may operate as an interface between the payor and benefit processors, on behalf of the payor. In at least one embodiment, the providers or representatives may be pharmacies. For example, as shown in FIG. 1, the representatives may include a first pharmacy (e.g., Pharmacy A) and a second pharmacy (e.g., Pharmacy B), and a first PBM (e.g., PBM A). Pharmacy A and Pharmacy B may be communicatively linked to the central system 102 to allow the pharmacies to transmit claim requests to the central system 102. For example, the pharmacies may be coupled to the central system 102 via a remote wireless connection or may communicate with the central system 102 through the Internet. The claim requests may represent requests to access and utilize benefits offered through benefit processors, such as a first PBM (e.g., PBM A) and a second PBM (e.g., PBM B), as shown in FIG. 1. The benefit processors may be similarly communicatively linked to the central system 102 via remote wireless connections, over the Internet, etc. In at least one embodiment, the claim request may be generated at one or more of Pharmacy A or Pharmacy B in response to a request from a client to access a benefit at the pharmacy. The client may be a payor (e.g., an employer providing health benefits to employees), or an associated payee (e.g., an employee). As an example, the claim request may be a prescription to be filled by the pharmacy. Relationships (e.g., arrangements) between the payor and the benefit processors, which may correspond to indirect relationships between a payee and the benefit processors, as defined by the payor contracts 110, may determine how the prescription is to be filled by the pharmacy, including prescription availability, amount of an expendable resource associated with the prescription, such as prescription costs, and applicable discounts provided by a benefit processor.

In at least one embodiment, the central system 102 may be an intermediary between the pharmacies and the benefit processors. As an example, the central system 102 may act as a central hub that relays information between the pharmacies and the benefit processors while receiving, compiling, assessing, and delivering data among the plurality of entities. In at least one embodiment, operations carried out by the central system 102 may be performed in real time. Furthermore, as described above, the central system 102 may assign a unique, permanent nexus identifier to a specific payor. In at least one embodiment, the nexus identifier may be a BIN/PCN (e.g., including the BIN ZZZ) that allows the payor to be identified by the central system 102 when a claim request is submitted on behalf of the payor (or payee). For example, the claim request may include the nexus identifier assigned to the payor and, upon receiving the claim request, the central system 102 may use the nexus identifier to generate a response to be returned to the pharmacy submitting the claim request.

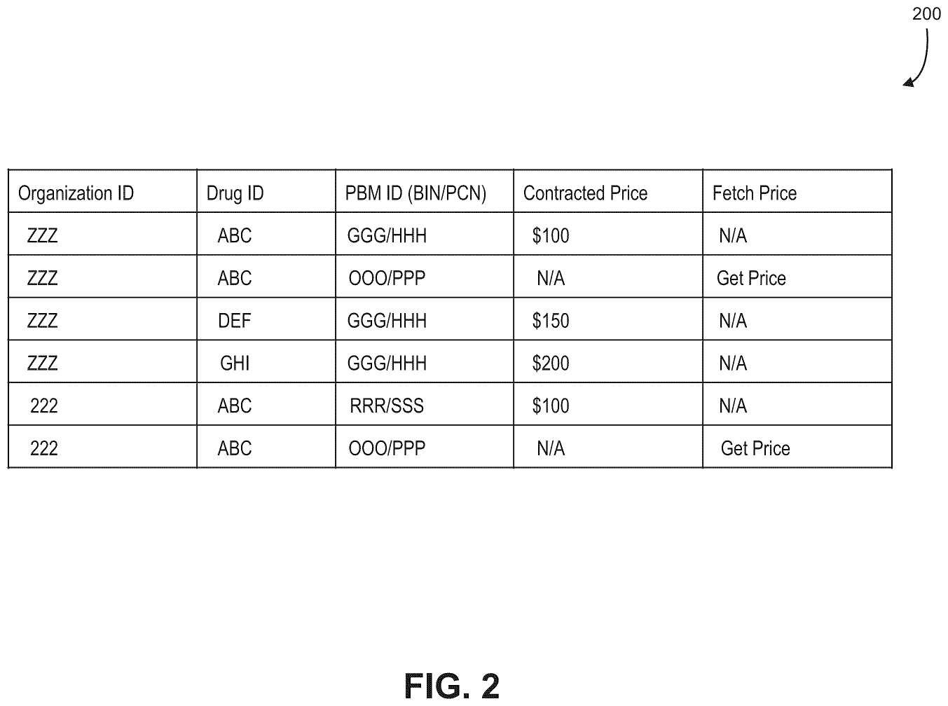

In at least one embodiment, a claim request submitted by Pharmacy A or Pharmacy B may be received by the central system 102, which may cause the customer module 104 to generate a mapping of relevant relationships, as shown in FIG. 2 and described further below. The relevant relationships may correspond to agreements between the payor and benefit processors and the mapping may be generated based on information provided in the claim request and data extracted from the payor contracts 110. As an example, the claim request may indicate a nexus identifier of the payor on whose behalf the claim request is submitted and what is being requested (e.g., what drug). The customer module 104 may implement commands (e.g., computer-executable instructions) to identify benefit processors that may provide a benefit corresponding to the claim request. The mapping may be generated to provide a comparison of relevant parameters offered by the identified benefit processors.

In at least one embodiment, in addition to mapping payor/benefit processor relationships, one or more requests or calls to obtain information relevant to the claim request may be triggered and/or submitted in response to receiving the claim request. For example, the information may include parameter values, such as a price of a drug. The parameter values may further include patient-specific prescription benefit information such as medication coverage, number of refills, alternative options, restrictions, an amount of an expendable resource associated with a requestable perquisite, including out-of-pocket costs, etc. In at least one embodiment, however, if a parameter value is fixed according to contract terms, e.g., fixed drug prices, the fixed values may be retrieved from the database 108. In another embodiment, a call may be a Real-Time Prescription Benefit (RTPB) call that allows a healthcare provider to obtain parameter values in real time. In other embodiments, however, the calls may be other types of requests depending on the type of information relevant to the request.

The one or more calls or requests to obtain information may be directed to one or more of PBM A and PBM B according to routing processing 112, which may include implementation of algorithms to route the calls to appropriate destinations (e.g., to a specific benefit processor). It will be appreciated that additional benefit processors may be communicatively linked to the central system 102 and only two benefit processors are depicted in FIG. 1 for brevity. In at least one embodiment, a benefit processor receiving the call, such as PBM A or PBM B, may return a price to the central system 102 in response to the call. One or more prices may thereby be received by the central system 102 and the prices may be provided to the customer module 104 to populate, e.g., input as entries, run-time transaction logic implemented thereat.

In at least one embodiment, the transaction logic executed at the customer module 104 may include mapping the payor, based on the assigned nexus identifier of the payor (e.g., ZZZ), to all benefit processors that the payor has an established relationship with. For example, the nexus identifier of the payor may be mapped to BIN/PCN pairs of all benefit processors that the payor is engaged with according to the payor contracts 110 and to details of the claim request, such as the type of drug. As an example, the BIN of PBM A may be GGG and the BIN of PBM B may be OOO, as indicated in FIG. 1.

An example is shown in FIG. 2 of a mapping 200 of a nexus identifier of a payor to a set of contracted benefit processors, where the mapping 200 may be generated in response to a claim request. The mapping 200 is depicted illustratively as a table but, in other examples, may be formatted or organized according to various formats and organizational schemes. In at least one embodiment, the mapping 200 may map the nexus identifier (e.g., a portion of which is the BIN ZZZ) of the payor to a set of benefit processors, the set including a drug identifier (ID), BIN/PCN pairs identifying the benefit processors, contracted pricing, and fetch pricing, which are depicted as columns of the mapping 200. In at least one embodiment, the BIN/PCN pairs of the benefit processors identify PBMs, including PBM A and PBM B of FIG. 1. Furthermore, when the mapping 200 is initially generated, e.g., in response to receiving a claim request, at least a portion of the mapping 200 may be unpopulated (e.g., empty). For example, column fields corresponding to the Contracted Price and Fetch Price columns in the mapping 200 may be initially unpopulated until responses are received from the relevant benefit processors.

The nexus identifier ZZZ may be correlated to drug identifiers representing drugs for which benefits may be provided by the benefit processors. For example, the drug identifiers may include ABC, DEF, and GHI, which may each represent a different drug type. The drug identifiers may be correlated to a BIN/PCN of a PBM providing a benefit relevant to the requested drug type. For example, the BIN/PCN pairs listed in the mapping 200 may include the BIN of PBM A (e.g., GGG) as well as a PCN, such as HHH, that identifies a group or subdivision within PBM A. The BIN/PCN pairs may also include the BIN of PBM B (e.g., OOO) as well as a PCN, such as PPP, that identifies a group or subdivision within PBM B. The BIN/PCN pairs further include additional BINs of other benefit processors (such as RRR/SSS). The drug identifiers and BIN/PCN may be further correlated to a contracted price, if available, that may be defined by a contract between the payor and the benefit processor. In some examples, such as for the payor BIN ZZZ, drug ID ABC, and PBM ID OOO/PPP, a contracted price may not be available. In such examples, a fetch price may be obtained instead, which may reflect a current market price of the drug.

The mapping 200 may also include an additional nexus identifier, such as 222, which may correspond to another payor that also has established contracts with at least one PBM in common with payor ZZZ. Multiple benefit processors may therefore be associated with a nexus identifier through the implementation of transaction logic at the customer module 104 of FIG. 1. Instead of directly routing a claim request to a BIN/PCN pair selected or provided by a payor, the claim request may be processed by the central system 102, with reference to FIG. 1, to map the nexus identifier to all BIN/PCN pairs that are matched to established contracts between the payor and benefit processors.

Returning to FIG. 1, in at least one embodiment, when the run-time transaction logic is implemented at the customer module 104 to map the nexus identifier to corresponding benefit processors, drug price requests may first be transmitted to the benefit processors. For example, as indicated in FIG. 1, drug price requests may be sent to PBM A and PBM B, which may return drug price responses indicating contracted or fetch prices for a requested drug. The drug price responses may be used to populate or input into the mapping (e.g., the mapping 200 of FIG. 2) with drug prices (contracted or fetch prices) and the mapping may be further processed according to price processing 114. In at least one embodiment, populating the mapping may include inputting, adding, entering, or inserting information into fields of the mapping where variables are located. In at least one embodiment, price processing 114 may include implementation of algorithms to apply rules defined by an entity managing and operating the central system 102. As an example, preset instructions or predetermined business rules may be applied to the prices provided by the selected benefit processor. Additional parameters may also be applied to the prices based on rules identified from the payor contracts 110, such as further requirements or modifications to the drug pricing.

Upon processing the mapping via price processing 114, a benefit processor, e.g., PBM A or PBM B, may be selected based on a comparison of prices for a requested drug. In at least one embodiment, a benefit processor offering the lowest drug price may be selected. In other embodiments, however, the benefit processor may be selected according to other criteria, such as availability of the drug, source or manufacturer, discounts offered in combination with other drug requests, etc. The selection of the benefit processor may be provided to the claims module 106.

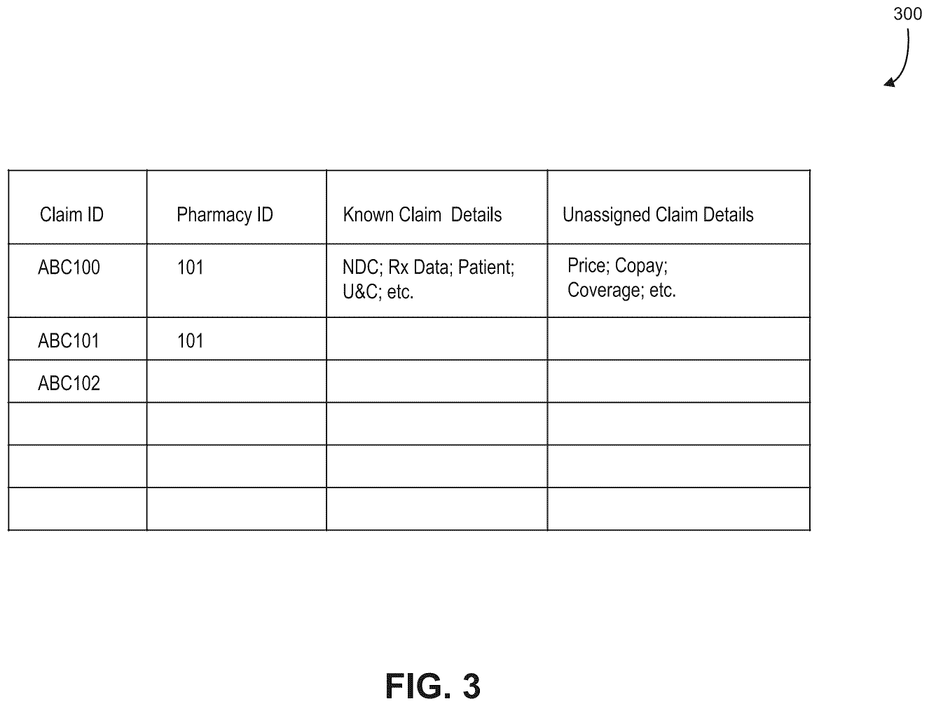

In at least one embodiment, the claims module 106 may be configured (e.g., with algorithms) to send the claim request to the selected benefit processor. For example, as indicated in FIG. 1, the claim request may be provided to one or more of PBM A or PBM B. The selected benefit processor may return a claim response to the central system 102, which may be received by the claims module 106. In at least one embodiment, the claim response may include information regarding the claim, which may be used to populate a relationship mapping, as illustrated in FIG. 3, at the claims module 106.

A relationship mapping 300 implemented by a claims module, e.g., the claims module 106 of FIG. 1, is shown in FIG. 3. The relationship mapping 300 may be populated using information received from the selected benefit processor regarding the claim request. In at least one embodiment, the relationship mapping 300 may include one or more claim identifiers identifying a specific claim request. For example, the claim request may include a claim identifier, such as ABC100, ABC101, ABC102, as shown in the relationship mapping 300, as well as a pharmacy identifier, e.g., 101, identifying a pharmacy from which the claim request originated. A claim response from a benefit processor may include information either provided by the claim request or available in records of the benefit processor, e.g., known claim details, that may be mapped to a specific claim identifier. The known claim details may include one or more of a National Drug Code (NDC) for the requested drug, prescription (e.g., Rx) data including dosage, quantity, and instructions for use, patient identification, a Usual and Customary (U&C) price of the requested drug, among other drug and/or prescription information.

In at least one embodiment, the relationship mapping may further include unassigned claim details corresponding to a specific claim identifier. The unassigned claim details may include parameters such as drug price, copayments, health insurance coverage, etc., which may not be provided in the claim request. Instead, the unassigned claim details may be obtained from claim responses received from one or more benefit processors (e.g., as shown in FIG. 1 and described above). The claims module thereby records information from claim responses provided by the benefit processors with respect to a claim request and maps the information to the known claim details of the claim request. A claim response may be generated by the claims module based on the relationship mapping 300.

In at least one embodiment, as shown in FIG. 1, a claim response generated by the claims module 106 may be returned to one or more of the pharmacies, e.g., Pharmacy A and/or Pharmacy B. The claim response may include at least benefits corresponding to the requested drug according to the outcome generated at the central system 102 (e.g., by the mapping 200 of FIG. 2 and the relationship mapping 300 of FIG. 3) and the unassigned claim details of the relationship mapping performed by the claims module 106. The claim response, therefore, provides the requesting pharmacy with information regarding a benefit provided by a benefit processor that is selected based on a comparison of available benefit options offered by available benefit processors. By assigning the nexus identifier to the payor, the nexus identifier may be associated with multiple PBMs, health insurance providers, benefit plans, or any other type of intermediary operating as a benefit processor. The payor may be alleviated of the burden of determining which benefit processor (e.g., BIN/PCN pair) to select, which may be especially challenging in instances where the payor has relationships with multiple benefit processors. In at least one embodiment, the most suitable benefit processor may include a benefit processor offering one or more benefits meeting, matching, satisfying, or otherwise fulfilling one or more criteria, such as lowest drug price, lowest copayment, highest coverage, etc., according to preset rules or instructions (also referred to herein as precedence logic). For example, the precedence logic may include rules or instructions to select a benefit processor that offers a lowest cost to a client for a drug, which may be a combination of lowest drug cost and lowest copayment. In another example, the precedence logic may include rules or instructions to select a benefit processor that offers highest coverage for a combination of two drugs. In yet another example, the precedence logic may include rules or instructions to select a benefit processor that offers a lowest drug price for a drug produced by a specific manufacturer. The architecture 100 further allows a payor with more than one claim request submission to obtain outcomes obtained from combinations of one or more benefit processors and/or one or more groups within one or more benefit processors.

In at least one embodiment, the architecture 100 of FIG. 1 may be similarly used to process a claim request from a payee (e.g., employee) accessing benefits defined by the payor contracts 110, where the payee may be associated with the payor (e.g., as an employee paying into a benefit plan through an employer). The payee may be similarly assigned a unique, durable identifier, such as a BIN/PCN, and the identifier of the payee may be linked to the nexus identifier of the payor. In at least one embodiment, the nexus identifier may be linked to multiple payee identifiers. For example, a mapping of a nexus identifier to payee identifiers may be input into the central system 102 during onboarding of the payor and stored there, e.g., at the database 108. The payor/payee mapping may be retrieved when a claim request is received that corresponds to one or more of the nexus identifier and/or payee identifier to provide contextual information regarding contractual relationships between the payor and payee that may further affect generation of an outcome in response to the claim request.

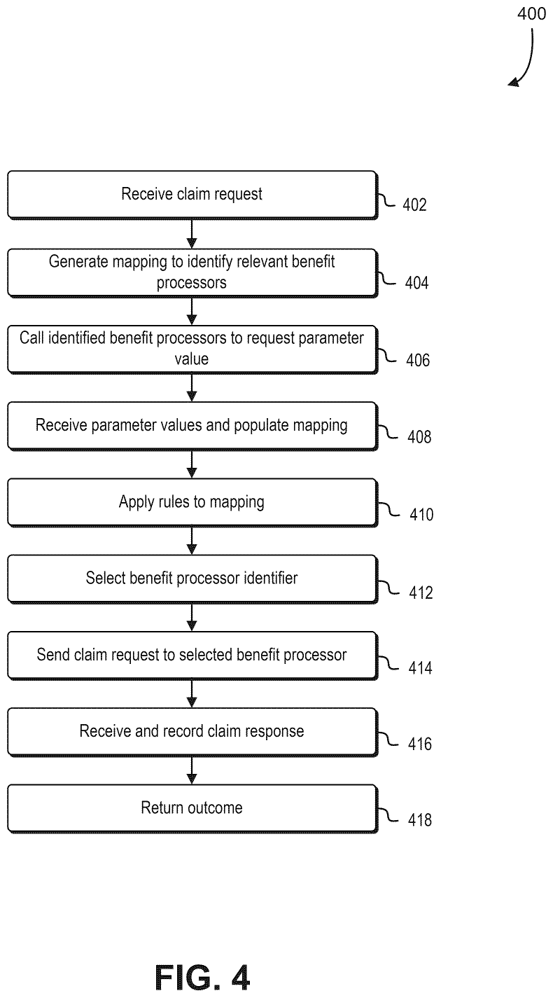

FIG. 4 is a flowchart illustrating an example of a process 400 for processing a claim request in accordance with various embodiments. Some or all of the process 400 (or any other processes described, or variations and/or combinations of those processes) may be performed under the control of one or more computer systems configured with executable instructions and/or other data and may be implemented as executable instructions executing collectively on one or more processors. The executable instructions and/or other data may be stored on a non-transitory computer-readable storage medium (e.g., a computer program persistently stored on magnetic, optical, or flash media). For example, some or all of process 400 may be performed by any suitable system, such as the computing device 500 of FIG. 5. In at least one embodiment, the process 400 may be performed at a system, such as the central system 102 of FIG. 1, that includes a customer module, a claims module, and a database, such as the customer module 104, claims module 106, and database 108 of FIG. 1. Furthermore, in at least one embodiment, a module may include one or more of software or hardware for implementing one or more algorithms for performing a set of operations at the module. The process 400 includes a series of operations which may involve storing and parsing documents, such as contracts, mapping relationships between a nexus identifier and identifiers of benefit providers (e.g., benefit processors) based on the documents and identifying the most suitable benefit providers for meeting or fulfilling a request according to preset rules such as precedence logic.

At 402, the process 400 may include receiving a claim request at the customer module of the central system, the claim request including a request for access to one or more benefits and a nexus (or payee) identifier. For example, the request may be submitted by a pharmacy on behalf of the payor or payee. At 404, in response to receiving the claim request, contract data stored at a database of the central system may be retrieved and parsed, based on the nexus identifier, and benefit providers offering benefits relevant to fulfilling the claim requests may be identified from the contract data. The nexus identifier may thereby be mapped to identifiers (e.g., BIN/PCNs) of relevant benefit providers by generating a mapping such as the mapping 200 of FIG. 2, and the mapping may indicate parameter values to be obtained from the benefit providers.

At 406, the process 400 may include calling the identified benefit providers to request parameter values corresponding to the claim request. For example, RTPB calls may be transmitted to the benefit providers to obtain target parameter values, such as a fetch price for a drug. The parameter values (e.g., drug price) may be received at 408 of the process 400 and used to populate unpopulated fields of the mapping according to run-time transaction logic implemented at the customer module.

At 410, the process 400 may include applying rules to the parameter values populating the mapping. For example, the rules may correspond to terms and conditions of an agreement between the payor and an entity managing and operating the central system. Additional rules that may be applied to the parameter values may include terms and conditions of a contract between the payor and a respective benefit provider that may impose specific modifications to the parameter values.

At 412, the process 400 may include selecting an identifier of a benefit provider according to the parameter values provided in the mapping and precedence logic implemented at the customer module. For example, the precedence logic may include instructions to select a BIN/PCN of a benefit provider offering the lowest drug price, a combination of lowest drug price and/or lowest copayment, a lowest drug price from a target drug manufacturer, or some other criterion or combination of criteria, as described above.

At 414, the process 400 may include sending the claim request to the selected BIN/PCN of the benefit provider. For example, the selected BIN/PCN may be provided to the claims module, which may generate a relationship mapping, such as the relationship mapping 300 of FIG. 3. In at least one embodiment, the relationship mapping 300 may be generated by parsing contractual data stored at the database that corresponds to one or more contracts between the payor and the benefit provider (e.g., the benefit provider identified by the selected BIN/PCN). The retrieved data may be used to populate the relationship mapping 300 with confirmed information relevant to the claim request, such as patient information, prescription information, etc., and with unassigned information, such as information provided by the benefit provider when called. The claim request may be sent with data from the relationship mapping, and the benefit provider may process the claim request and accompanying data to generate a claim response. For example, the parameter value previously received from the benefit provider may be confirmed or updated by the benefit provider based on the information provided with the claim request and returned in the claim response.

At 416, the process 400 may include receiving and recording the claim response from the benefit provider. For example, details of the received claim response may be recorded and stored in the database of the central system in conjunction with data from the contracts between the payor and the benefit providers.

At 418, the process 400 may include returning an outcome to the pharmacy that submitted the claim request. For example, the outcome may include the claim response received from the benefit provider corresponding to the selected benefit provider identifier. Note that one or more of the operations performed in 402-418 may be performed in various orders and combinations, including in parallel.

Note that, in the context of describing disclosed embodiments, unless otherwise specified, use of expressions regarding executable instructions (also referred to as code, applications, agents, etc.) performing operations that “instructions” do not ordinarily perform unaided (e.g., transmission of data, calculations, etc.) denotes that the instructions are being executed by a machine, thereby causing the machine to perform the specified operations.

FIG. 5 is an illustrative, simplified block diagram of the computing device 500 that can be used to practice at least one embodiment of the present disclosure. In at least one embodiment, the computing device 500 may be used to perform the operations and processes described above with respect to the central system 102 of FIG. 1. In various embodiments, the computing device 500 includes any appropriate device operable to send and/or receive requests, messages, or information over an appropriate network and convey information back to a user of the device. The computing device 500 may be used to implement any of the systems illustrated and described above. For example, the computing device 500 may be configured for use as a data server, a web server, a portable computing device, a personal computer, a cellular or other mobile phone, a handheld messaging device, a laptop computer, a tablet computer, a set-top box, a personal data assistant, an embedded computer system, an electronic book reader, or any electronic computing device. The computing device 500 may be implemented as a hardware device, a virtual computer system, or one or more programming modules executed on a computer system, and/or as another device configured with hardware and/or software to receive and respond to communications (e.g., web service application programming interface (API) requests) over a network.

As shown in FIG. 5, the computing device 500 may include one or more processors 502 that, in embodiments, communicate with and are operatively coupled to a number of peripheral subsystems via a bus subsystem. In some embodiments, these peripheral subsystems include a storage subsystem 506, comprising a memory subsystem 508 and a file/disk storage subsystem 510, one or more user interface input devices 512, one or more user interface output devices 514, and a network interface subsystem 516. Such storage subsystem 506 may be used for temporary or long-term storage of information.

In some embodiments, the bus subsystem 504 may provide a mechanism for enabling the various components and subsystems of computing device 500 to communicate with each other as intended. Although the bus subsystem 504 is shown schematically as a single bus, alternative embodiments of the bus subsystem utilize multiple buses. The network interface subsystem 516 may provide an interface to other computing devices and networks. The network interface subsystem 516 may serve as an interface for receiving data from and transmitting data to other systems from the computing device 500. In some embodiments, the bus subsystem 504 is utilized for communicating data such as details, search terms, and so on. In an embodiment, the network interface subsystem 516 may communicate via any appropriate network that would be familiar to those skilled in the art for supporting communications using any of a variety of commercially available protocols, such as Transmission Control Protocol/Internet Protocol (TCP/IP), User Datagram Protocol (UDP), protocols operating in various layers of the Open System Interconnection (OSI) model, File Transfer Protocol (FTP), Universal Plug and Play (UpnP), Network File System (NFS), Common Internet File System (CIFS), and other protocols.

The network, in an embodiment, is a local area network, a wide-area network, a virtual private network, the Internet, an intranet, an extranet, a public switched telephone network, a cellular network, an infrared network, a wireless network, a satellite network, or any other such network and/or combination thereof, and components used for such a system may depend at least in part upon the type of network and/or system selected. In an embodiment, a connection-oriented protocol is used to communicate between network endpoints such that the connection-oriented protocol (sometimes called a connection-based protocol) is capable of transmitting data in an ordered stream. In an embodiment, a connection-oriented protocol can be reliable or unreliable. For example, the TCP protocol is a reliable connection-oriented protocol. Asynchronous Transfer Mode (ATM) and Frame Relay are unreliable connection-oriented protocols. Connection-oriented protocols are in contrast to packet-oriented protocols such as UDP that transmit packets without a guaranteed ordering. Many protocols and components for communicating via such a network are well known and will not be discussed in detail. In an embodiment, communication via the network interface subsystem 516 is enabled by wired and/or wireless connections and combinations thereof.

In some embodiments, the user interface input devices 512 includes one or more user input devices such as a keyboard; pointing devices such as an integrated mouse, trackball, touchpad, or graphics tablet; a scanner; a barcode scanner; a touch screen incorporated into the display; audio input devices such as voice recognition systems, microphones; and other types of input devices. In general, use of the term “input device” is intended to include all possible types of devices and mechanisms for inputting information to the computing device 500. In some embodiments, the one or more user interface output devices 514 include a display subsystem, a printer, or non-visual displays such as audio output devices, etc. In some embodiments, the display subsystem includes a cathode ray tube (CRT), a flat-panel device such as a liquid crystal display (LCD), light emitting diode (LED) display, or a projection or other display device. In general, use of the term “output device” is intended to include all possible types of devices and mechanisms for outputting information from the computing device 500. The one or more user interface output devices 514 can be used, for example, to present user interfaces to facilitate user interaction with applications performing processes described and variations therein, when such interaction may be appropriate.

In some embodiments, the storage subsystem 506 provides a computer-readable storage medium for storing the basic programming and data constructs that provide the functionality of at least one embodiment of the present disclosure. The applications (programs, code modules, instructions), when executed by one or more processors in some embodiments, provide the functionality of one or more embodiments of the present disclosure and, in embodiments, are stored in the storage subsystem 506. These application modules or instructions can be executed by the one or more processors 502. In various embodiments, the storage subsystem 506 additionally provides a repository for storing data used in accordance with the present disclosure. In some embodiments, the storage subsystem 506 comprises a memory subsystem 508 and a file/disk storage subsystem 510.

In embodiments, the memory subsystem 508 includes a number of memories, such as a main random-access memory (RAM) 518 for storage of instructions and data during program execution and/or a read only memory (ROM) 520, in which fixed instructions can be stored. In some embodiments, the file/disk storage subsystem 510 provides a non-transitory persistent (non-volatile) storage for program and data files and can include a hard disk drive, a floppy disk drive along with associated removable media, a Compact Disk Read Only Memory (CD-ROM) drive, an optical drive, removable media cartridges, or other like storage media.

In some embodiments, the computing device 500 includes at least one local clock 524. The at least one local clock 524, in some embodiments, is a counter that represents the number of ticks that have transpired from a particular starting date and, in some embodiments, is located integrally within the computing device 500. In various embodiments, the at least one local clock 524 is used to synchronize data transfers in the processors for the computing device 500 and the subsystems included therein at specific clock pulses and can be used to coordinate synchronous operations between the computing device 500 and other systems in a data center. In another embodiment, the local clock is a programmable interval timer.

The computing device 500 could be of any of a variety of types, including a portable computer device, tablet computer, a workstation, or any other device described below. Additionally, the computing device 500 can include another device that, in some embodiments, can be connected to the computing device 500 through one or more ports (e.g., USB, a headphone jack, Lightning connector, etc.). In embodiments, such a device includes a port that accepts a fiber-optic connector. Accordingly, in some embodiments, this device converts optical signals to electrical signals that are transmitted through the port connecting the device to the computing device 500 for processing. Due to the ever-changing nature of computers and networks, the description of the computing device 500 depicted in FIG. 5 is intended only as a specific example for purposes of illustrating the preferred embodiment of the device. Many other configurations having more or fewer components than the system depicted in FIG. 5 are possible.

The specification and drawings are, accordingly, to be regarded in an illustrative rather than a restrictive sense. However, it will be evident that various modifications and changes may be made thereunto without departing from the scope of the invention as set forth in the claims. Likewise, other variations are within the scope of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed but, on the contrary, the intention is to cover all modifications, alternative constructions and equivalents falling within the scope of the invention, as defined in the appended claims.

In some embodiments, data may be stored in a data store (not depicted). In some examples, a “data store” refers to any device or combination of devices capable of storing, accessing, and retrieving data, which may include any combination and number of data servers, databases, data storage devices, and data storage media, in any standard, distributed, virtual, or clustered system. A data store, in an embodiment, communicates with block-level and/or object level interfaces. The computing device 500 may include any appropriate hardware, software, and firmware for integrating with a data store as needed to execute aspects of one or more applications for the computing device 500 to handle some or all of the data access and business logic for the one or more applications. The data store, in an embodiment, includes several separate data tables, databases, data documents, dynamic data storage schemes, and/or other data storage mechanisms and media for storing data relating to a particular aspect of the present disclosure. In an embodiment, the computing device 500 includes a variety of data stores and other memory and storage media as discussed above. These can reside in a variety of locations, such as on a storage medium local to (and/or resident in) one or more of the computers or remote from any or all of the computers across a network. In an embodiment, the information resides in a storage-area network (SAN) familiar to those skilled in the art, and, similarly, any necessary files for performing the functions attributed to the computers, servers or other network devices are stored locally and/or remotely, as appropriate.

In an embodiment, the computing device 500 may provide access to content including, but not limited to, text, graphics, audio, video, and/or other content that is provided to a user in the form of HyperText Markup Language (HTML), Extensible Markup Language (XML), JavaScript, Cascading Style Sheets (CSS), JavaScript Object Notation (JSON), and/or another appropriate language. The computing device 500 may provide the content in one or more forms including, but not limited to, forms that are perceptible to the user audibly, visually, and/or through other senses. The handling of requests and responses, as well as the delivery of content, in an embodiment, is handled by the computing device 500 using PHP: Hypertext Preprocessor (PHP), Python, Ruby, Perl, Java, HTML, XML, JSON, and/or another appropriate language in this example. In an embodiment, operations described as being performed by a single device are performed collectively by multiple devices that form a distributed and/or virtual system.

In an embodiment, the computing device 500 typically will include an operating system that provides executable program instructions for the general administration and operation of the computing device 500 and includes a computer-readable storage medium (e.g., a hard disk, random access memory (RAM), read only memory (ROM), etc.) storing instructions that if executed (e.g., as a result of being executed) by a processor of the computing device 500 cause or otherwise allow the computing device 500 to perform its intended functions (e.g., the functions are performed as a result of one or more processors of the computing device 500 executing instructions stored on a computer-readable storage medium).

In an embodiment, the computing device 500 operates as a web server that runs one or more of a variety of server or mid-tier applications, including Hypertext Transfer Protocol (HTTP) servers, FTP servers, Common Gateway Interface (CGI) servers, data servers, Java servers, Apache servers, and business application servers. In an embodiment, computing device 500 is also capable of executing programs or scripts in response to requests from user devices, such as by executing one or more web applications that are implemented as one or more scripts or programs written in any programming language, such as Java®, C, C #or C++, or any scripting language, such as Ruby, PHP, Perl, Python, or TCL, as well as combinations thereof. In an embodiment, the computing device 500 is capable of storing, retrieving, and accessing structured or unstructured data. In an embodiment, computing device 500 additionally or alternatively implements a database, such as one of those commercially available from Oracle®, Microsoft®, Sybase®, and IBM® as well as open-source servers such as MySQL, Postgres, SQLite, MongoDB. In an embodiment, the database includes table-based servers, document-based servers, unstructured servers, relational servers, non-relational servers, or combinations of these and/or other database servers.

A system of one or more computers may be configured to perform particular operations or actions by virtue of having software, firmware, hardware, or a combination of them installed on the system that in operation causes or cause the system to perform the actions. One or more computer programs may be configured to perform particular operations or actions by virtue of including instructions that, when executed by data processing apparatus, cause the apparatus to perform the actions. One general aspect includes a computer-implemented method. The computer-implemented method includes generating a client identifier for a client and generating, in response to receiving a request for the client to access a requestable perquisite, a mapping of the client identifier to one or more identifiers corresponding to a set of providers able to meet the request. The computer-implemented method also includes inputting, into the mapping, values from the set of providers associated with the requestable perquisite, selecting a provider from the set of providers based, at least in part, on the mapping and preset instructions, and providing the request to the selected provider to obtain a response from the selected provider. In addition, the computer-implemented method includes providing the response from the selected provider as a result of processing the received request. Other embodiments of this aspect include corresponding computer systems, apparatus, and computer programs recorded on one or more computer storage devices, each configured to perform the actions of the methods.

Implementations of the computer-implemented may include storing one or more documents defining a set of conditions for the client and the set of providers; and the values are determined from the one or more documents. In another implementation, the computer-implemented method may include receiving the request from a provider of the requestable perquisite; and the response is returned to the provider of the requestable perquisite. In another implementation, the mapping is generated at a first component of a profile mapping system, where the first component performs operations to identify the values based, at least in part, on the mapping. In yet another implementation, an additional mapping is generated at a second component of a profile mapping system, and the second component performs operations to map the request to information corresponding to one or more documents defining a set of conditions for the client and the selected provider. The information is provided to the selected provider with the request. Implementations of the described techniques may include hardware, a method or process, or computer software on a computer-accessible medium.

Another general aspect includes a system that includes one or more processors. The system also includes memory including computer-executable instructions that, if executed by the one or more processors, cause the system to generate a client identifier for a client, generate, in response to receiving a request corresponding to the client identifier to access a requestable perquisite, a mapping of the client identifier to one or more identifiers corresponding to a set of providers able to meet the request, input, into the mapping, values associated with the requestable perquisite from the set of providers, and select a provider from the set of providers based, at least in part, on the mapping and preset instructions. The computer-executable instructions, if executed by the one or more processors, further cause the system to provide the request to the selected provider to receive a response from the selected provider, and provide the response from the selected provider as a result of processing the received request. Other embodiments of this aspect include corresponding computer systems, apparatus, and computer programs recorded on one or more computer storage devices, each configured to perform the actions of the methods.

Implementations may include one or more of the following features. The computer-executable instructions may also include instructions that cause the system to submit requests to the set of providers for the values. The computer-executable instructions may further include instructions that cause the system to identify the values from a database storing fixed values. The computer-executable instructions that cause the system to identify the values may further include instructions to apply conditions extracted from documents representing arrangements between the client and the set of providers to the values to update the values. The computer-executable instructions that cause the system to select the selected provider may further include instructions that cause the system to use the preset instructions to identify at least one of the values added to the mapping that satisfies the preset instructions. In one implementation, the client identifier does not correspond to any of the one or more identifiers corresponding to the set of providers. In another implementation, the client identifier remains active when a new document defining a new set of conditions for the client and a new provider in addition to the set of providers is stored, and when an arrangement between the client and a provider of the set of providers is terminated. In yet another implementation, the values may include one or more parameters affecting an amount of an expendable resource associated with acquiring the requestable perquisite. Implementations of the described techniques may include hardware, a method or process, or computer software on a computer-accessible medium.

One general aspect includes a non-transitory computer-readable storage medium having stored thereon executable instructions that, if executed by one or more processors of a computer system, cause the computer system to at least cause a client identifier of a client to be mapped to identifiers corresponding to a set of providers according to conditions defined by arrangements between the client and the set of providers. The executable instructions, if executed, also causes the computer system to use the mapping of the client identifier to the identifiers to compare one or more values provided by the set of providers, the one or more values corresponding to a requestable perquisite requested by the client, and select a provider from the set of providers based, at least in part, on the comparison. Other embodiments of this aspect include corresponding computer systems, apparatus, and computer programs recorded on one or more computer storage devices, each configured to perform the actions of the methods.

Implementations may include one or more of the following features. The executable instructions may further include instructions that cause the computer system to assign the client identifier to the client and use the client identifier to identify the set of providers from data stored at a database. The executable instructions may also include instructions that cause the computer system to link additional client identifiers to the client identifier, the additional client identifiers corresponding to additional clients requesting access to requestable perquisites moderated by the set of providers. In one implementation, a request to access the requestable perquisite requested by the client is provided to the computer system by a provider of the requestable perquisite. In another implementation, the set of providers may include entities that moderate how the requestable perquisite is provided to the client. The executable instructions that cause the computer system to map the client identifier to the identifiers corresponding to the set of providers may also include instructions that cause the computer system to generate the mapping in response to receiving a request on behalf of the client to access the requestable perquisite, and to compare the values to identify which of the set of providers meets a set of conditions for generating a response to the request. In one implementation, the arrangements are determined from documents stored at a database, the documents defining a set of conditions for the client and the set of providers; and the identifiers corresponding to the set of providers indicate how the requestable perquisite is to be distributed based, at least in part, on the documents. Implementations of the described techniques may include hardware, a method or process, or computer software on a computer-accessible medium.

The use of the terms “a” and “an” and “the” and similar referents in the context of describing the disclosed embodiments (especially in the context of the following claims) is to be construed to cover both the singular and the plural, unless otherwise indicated or clearly contradicted by context. The terms “comprising,” “having,” “including” and “containing” are to be construed as open-ended terms (i.e., meaning “including, but not limited to,”) unless otherwise noted. The term “connected,” when unmodified and referring to physical connections, is to be construed as partly or wholly contained within, attached to, or joined together, even if there is something intervening. Recitation of ranges of values in the present disclosure are merely intended to serve as a shorthand method of referring individually to each separate value falling within the range unless otherwise indicated and each separate value is incorporated into the specification as if it were individually recited. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal. The use of the phrase “based on,” unless otherwise explicitly stated or clear from context, means “based at least in part on” and is not limited to “based solely on.”

Conjunctive language, such as phrases of the form “at least one of A, B, and C,” or “at least one of A, B and C,” unless specifically stated otherwise or otherwise clearly contradicted by context, is otherwise understood with the context as used in general to present that an item, term, etc., could be either A or B or C, or any nonempty subset of the set of A and B and C. For instance, in the illustrative example of a set having three members, the conjunctive phrases “at least one of A, B, and C” and “at least one of A, B, and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present.

Operations of processes described can be performed in any suitable order unless otherwise indicated or otherwise clearly contradicted by context. Processes described (or variations and/or combinations thereof) can be performed under the control of one or more computer systems configured with executable instructions and can be implemented as code (e.g., executable instructions, one or more computer programs or one or more applications) executing collectively on one or more processors, by hardware or combinations thereof. In some embodiments, the code can be stored on a computer-readable storage medium, for example, in the form of a computer program comprising a plurality of instructions executable by one or more processors. In some embodiments, the computer-readable storage medium is non-transitory.

The use of any and all examples, or exemplary language (e.g., “such as”) provided, is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described, including the best mode known to the inventors for carrying out the invention. Variations of those embodiments will become apparent to those of ordinary skill in the art upon reading the foregoing description. The inventors expect skilled artisans to employ such variations as appropriate and the inventors intend for embodiments of the present disclosure to be practiced otherwise than as specifically described. Accordingly, the scope of the present disclosure includes all modifications and equivalents of the subject matter recited in the claims appended hereto as permitted by applicable law. Moreover, any combination of the above-described elements in all possible variations thereof is encompassed by the scope of the present disclosure unless otherwise indicated or otherwise clearly contradicted by context.

All references, including publications, patent applications, and patents, cited are hereby incorporated by reference to the same extent as if each reference were individually and specifically indicated to be incorporated by reference and were set forth in its entirety.

1. A computer-implemented method performed by a centralized computing system to link client identifiers to providers of a requested perquisite in real time, the computer-implemented method comprising:
providing remote access to a set of providers over a network;
receiving, from a client, a request to access a requestable perquisite;
retrieving a set of conditions associated with the client and the set of providers;
generating a client identifier for the client;
generating, via run-time transaction logic, a mapping that correlates the client identifier to one or more provider identifiers corresponding to a subset of providers, of the set of providers, able to meet the request in accordance with the set of conditions;
inputting, into the mapping, values retrieved in real time from the subset of providers, each value associated with the requestable perquisite and a provider offering the requestable perquisite, the inputting performed according to the run-time transaction logic;
selecting a provider from the subset of providers based, at least in part, on one or more comparisons between the values in the mapping;
providing the request and the set of conditions to the selected provider to obtain a response, from the selected provider, that is specific to the client identifier; and
providing the response from the selected provider as a result of processing the received request.

2. The computer-implemented method of claim 1, wherein:
the computer-implemented method further comprises storing one or more documents defining the set of conditions; and
the values are determined from the one or more documents.

3. (canceled)

4. The computer-implemented method of claim 1, wherein the mapping is generated at a first component of a profile mapping system, wherein the first component performs operations to identify the values based, at least in part, on the mapping.

5. The computer-implemented method of claim 1, further comprising generating an additional mapping at a second component of a profile mapping system, wherein the second component performs operations to map the request to information corresponding to one or more documents defining the set of conditions, and wherein the information is provided to the selected provider with the request.

6. A centralized computing system, comprising:
one or more processors; and
memory including computer-executable instructions that, if executed by the one or more processors, cause the centralized computing system to:
provide remote access to a set of providers over a network;
receive, from a client, a request to access a requestable perquisite;
retrieve a set of conditions associated with the client and the set of providers;
generate a client identifier corresponding to the client;
generate, via run-time transaction logic, a mapping that correlates the client identifier to one or more provider identifiers corresponding to a subset of providers, of the set of providers, able to meet the request in accordance with the set of conditions;
input, into the mapping, values retrieved in real time from the subset of providers, each value associated with the requestable perquisite and a provider offering the requestable perquisite, the inputting performed according to the run-time transaction logic;
select a provider from the subset of providers based, at least in part, on one or more comparisons between the values in the mapping;
provide the request and the set of conditions to the selected provider to receive a response, from the selected provider, that is specific to the client identifier; and
provide the response from the selected provider as a result of processing the received request.

7. The centralized computing system of claim 6, wherein the computer-executable instructions further include instructions that cause the centralized computing system to submit requests to the subset of providers for the values.

8. The centralized computing system of claim 6, wherein the computer-executable instructions further include instructions that cause the centralized computing system to identify the values from a database storing fixed values.

9. The centralized computing system of claim 6, wherein the computer-executable instructions further include instructions that cause the centralized computing system to apply the set of conditions to the values to update the values, wherein the set of conditions are extracted from documents representing arrangements between the client and the set of providers.

10. The centralized computing system of claim 6, wherein the computer-executable instructions that cause the centralized computing system to select the provider from the subset of providers further include instructions that cause the centralized computing system to identify at least one of the values added to the mapping that satisfies preset instructions.

11. The centralized computing system of claim 6, wherein the client identifier does not correspond to any of the one or more provider identifiers.

12. The centralized computing system of claim 6, wherein the client identifier remains active when:
a new document defining a new set of conditions for the client and a new provider in addition to the set of providers is stored; or
an arrangement between the client and a provider of the set of providers is terminated.

13. The centralized computing system of claim 6, wherein the values comprise one or more parameters affecting an amount of an expendable resource associated with acquiring the requestable perquisite.

14. A non-transitory computer-readable storage medium having stored thereon executable instructions that, if executed by one or more processors of a centralized computing computer system, cause the centralized computing system to at least:
provide remote access to a set of providers over a network;
receive a request on behalf of a client to access a requestable perquisite;
cause, via run-time transaction logic, a client identifier of a client to be mapped to one or more provider identifiers corresponding to a subset of providers, of the set of providers, able to meet the request according to a set of conditions defined by arrangements between the client and the set of providers;
use the mapping of the client identifier to the one or more provider identifiers to compare one or more values provided in real time by the subset of providers, each of the one or more values corresponding to the requestable perquisite and a provider offering the requestable perquisite; and
select a provider from the subset of providers based, at least in part, on the comparison, the selected provider to provide access to the requestable perquisite.

15. The non-transitory computer-readable storage medium of claim 14, wherein the executable instructions further include instructions that cause the centralized computing system to:
assign the client identifier to the client; and
use the client identifier to identify the subset of providers from data stored at a database.

16. The non-transitory computer-readable storage medium of claim 14, wherein the executable instructions further include instructions that cause the centralized computing system to link additional client identifiers to the client identifier, the additional client identifiers corresponding to additional clients requesting access to requestable perquisites moderated by the set of providers.

17. The non-transitory computer-readable storage medium of claim 14, wherein the request on behalf of the client to access the requestable perquisite is provided to the centralized computing system by a provider of the requestable perquisite.

18. The non-transitory computer-readable storage medium of claim 14, wherein the subset of providers comprise entities that moderate how the requestable perquisite is to be provided to the client.

19. The non-transitory computer-readable storage medium of claim 14, wherein:
the executable instructions that cause the centralized computing system to cause the client identifier to be mapped to the one or more provider identifiers include instructions that cause the centralized computing system to:
generate the mapping in response to receiving the request on behalf of the client to access the requestable perquisite; and
identify which of the subset of providers is able to generate a response to the request.

20. The non-transitory computer-readable storage medium of claim 14, wherein:
the arrangements are determined from documents stored at a database; and
the one or more provider identifiers indicate how the requestable perquisite is to be distributed based, at least in part, on the documents.

21. The non-transitory computer-readable storage medium of claim 14, wherein the executable instructions further include instructions that cause the centralized computing system to:
provide the request and the set of conditions to the selected provider to obtain a response, from the selected provider, that is specific to the client identifier; and
provide the response from the selected provider as a result of processing the request.
